(19) United States
(12) Patent Application Publication
(10) Pub. No.: US 2026/0295014 A1
(43) Pub. Date: Oct. 1, 2026

(54) HIGH BIOAVAILABILITY PHARMACEUTICAL COMPOSITION OF GLP AGONIST FOR ORAL ADMINISTRATION

(71) Applicants: Siegfried GSCHLIESSER, Taipei City (TW); Bhushan DESAI, Thane (IN); ANYA BIOPHARM INC., Taipei City (TW)

(72) Inventors: Siegfried GSCHLIESSER, Taipei City (TW); Bhushan DESAI, Thane (IN); Saurabh PATIL, Thane (IN)

(73) Assignee: ANYA BIOPHARM INC., Taipei City (TW)

(21) Appl. No.: 19/706,250

(22) Filed: Jun. 12, 2026

Related U.S. Application Data
(63) Continuation of application No. 19/164,672, filed on Mar. 6, 2024.

(30) Foreign Application Priority Data
Mar. 23, 2023 (IN) 202321020795

(51) Int. Cl. A61K 38/26; A61K 9/20

(52) U.S. Cl. CPC A61K 38/26; A61K 9/2009; A61K 9/2013; A61K 9/2095

(57) ABSTRACT

The present invention relates to a pharmaceutical composition for oral administration comprising a GLP receptor agonist or pharmaceutically acceptable salt or derivatives thereof, one or more medium chain fatty acid based permeation enhancers with aromatic functional group, and one or more medium chain fatty acid based permeation enhancers without aromatic functional group. The pharmaceutical composition may optionally further comprise a basifier.

### CROSS-REFERENCE TO RELATED APPLICATIONS

This application is a Continuation of U.S. application Ser. No. 19/164,672 filed on Sep. 12, 2025, which is a National Stage of International Application No. PCT/IB2024/052151 filed on Mar. 6, 2024, claiming priority based on Indian Patent Application number 202321020795 filed on Mar. 23, 2023. The disclosure of U.S. application Ser. No. 19/164,672 is incorporated herein by reference in its entirety.

The present invention relates to a pharmaceutical composition for oral administration comprising a GLP receptor agonist or pharmaceutically acceptable salt or derivatives thereof, one or more medium chain fatty acid based permeation enhancers with aromatic functional group, and one or more medium chain fatty acid based permeation enhancers without aromatic functional group. The pharmaceutical composition may optionally further comprise a basifier.

### BACKGROUND OF INVENTION

Peptides are large macromolecules mainly made up of amino acids (AAs) attached to each other. Peptides are the major therapeutic compounds used for the treatment of various disease or disorder. Peptides are majorly administered as injection which poses major patient compliance challenge as it is cumbersome for the patients requiring repeated administration.

There have been multiple attempts to delivery peptides by oral route. However, majority of them has been unsuccessful largely due to susceptibility of peptide to gastrointestinal tract (GIT) enzymes. Permeability of these large macromolecules from GIT poses further challenge to development of oral therapy for such macromolecules.

Therefore, there is an urgent need for the improved oral delivery therapy with pharmaceutical composition comprising peptides or pharmaceutically acceptable salts or derivatives thereof. It is surprisingly found that the composition as per invention can deliver peptide through oral route effectively.

### DESCRIPTION OF DRAWINGS

FIG. 1: Plasma concentration time profile curve for example 1 vs Rybelsus 7 mg tablets in human volunteers.

FIG. 2: Dissolution profile of the composition as per example 1 performed in Phosphate Buffer with pH 6.8.

### DETAILED DESCRIPTION OF THE INVENTION

The present invention relates to a pharmaceutical composition for oral administration comprising a GLP receptor agonist or pharmaceutically acceptable salt or derivatives thereof, one or more medium chain fatty acid based permeation enhancers with aromatic functional group, and one or more medium chain fatty acid based permeation enhancers without; aromatic functional group. The pharmaceutical composition may optionally further comprise a basifier.

“Therapeutically effective amount” or “effective amount” refers to the amount of a pharmaceutically active agent when administered, is sufficient to affect such prevention or treatment. The therapeutically effective amount will vary depending on the disease and its severity, and the age, weight, and other conditions of the patient to be treated. The pharmaceutical composition as per present invention are useful for the treatment of disease involving GLP receptors including but not limited to type 2 diabetes and obesity.

“Medium chain fatty acid” refers to fatty acids with C6 to C12 carbon chain. Examples of medium chain fatty acid includes caproic acid (C6), caprylic acid (C8), capric acid (C10), and lauric acid (C10).

In an embodiment, the present invention relates to a pharmaceutical composition for oral administration comprising
a. a therapeutically effective amount of GLP receptor agonist or pharmaceutically acceptable salt or derivatives thereof
b. one or more medium chain fatty acid based permeation enhancers with aromatic functional group, and
c. one or more medium chain fatty acid based permeation enhancers without aromatic functional group.

In an another embodiment, the present invention relates to a pharmaceutical composition for oral administration comprising
a. a therapeutically effective amount of GLP receptor agonist or pharmaceutically acceptable salt or derivatives thereof,
b. one or more medium chain fatty acid based permeation enhancers with aromatic functional group,
c. one or more medium chain fatty acid based permeation enhancers without aromatic functional group, and
d. one or more basifier.

In an another embodiment, the present invention relates to a pharmaceutical composition for oral administration comprising
a. a therapeutically effective amount of semaglutide,
b. one or more medium chain fatty acid based permeation enhancers with aromatic functional group,
c. one or more medium chain fatty acid based permeation enhancers without aromatic functional group, and
d. one or more basifier.

In a specific embodiment, medium chain fatty acid based permeation enhancers with aromatic functional group is Salcaprozate sodium. In a more specific embodiment, Salcaprozate sodium is in amount of 25 mg to 200 mg.

In an another specific embodiment, the present invention relates to a pharmaceutical composition for oral administration comprising
a. a therapeutically effective amount of semaglutide,
b. salcaprozate sodium,
c. sodium caprate or sodium caprylate or combinations thereof, and
d. calcium carbonate.

In an another embodiment, the pharmaceutical composition as per present invention is tablet and are prepared by direct compression method. In one embodiments, tablet does not contains binder.

In one or more embodiments, AUC₀₋ₜ (ngxh/mL) for one or more pharmaceutical composition as per present invention is 1.5 times or more compare to AUC₀₋ₜ (ngxh/mL) for reference product Rybelsus in single dose fasted study in human volunteers. In one or more embodiments, AUC₀₋ₜ (ngxh/mL) for one or more pharmaceutical composition as per present invention is 2 times or more compare to AUC₀₋ₜ (ngxh/mL) for reference product Rybelsus in single dose fasted study in human volunteers. In one or more embodiments, the ratio of AUC0-t (ngxh/mL) of reference product Rybelsus to AUC0-t (ngxh/mL) of one or more pharmaceutical composition as per present invention in single dose fasted study in human volunteers is 1:1.5 to 1:6.

In one or more embodiments, Cmax (ng/ml) for one or more pharmaceutical composition as per present invention is 1.5 times or more compare to Cmax (ng/mL) for reference product Rybelsus in single dose fasted study in human volunteers. In one or more embodiments, Cmax (ng/ml) for one or more pharmaceutical composition as per present invention is 2 times or more compare to Cmax (ng/mL) for reference product Rybelsus in single dose fasted study in human volunteers. In one or more embodiments, the ratio of Cmax (ng/ml) of reference product Rybelsus to Cmax (ng/ml) of one or more pharmaceutical composition as per present invention in single dose fasted study in human volunteers is 1:1.5 to 1:6.

### Glucagon-Like Peptide (GLP) Receptor Agonist

Glucagon-like Peptide (GLP) receptor agonist are agonists of the GLP receptor. GLP receptor agonist are class of compound majorly used for the treatment of type 2 diabetes. There are many other applications of GLP receptor agonist which includes weight management therapy. Without limiting, the examples of GLP receptor agonist includes Semaglutide, Exenatide, Liraglutide, Tirzepatide, Albiglutide, Dulaglutide, and Lixisenatide. The pharmaceutical composition as per present invention may contain therapeutically effective amount of GLP receptor agonist.

### Semaglutide

Semaglutide is a Glucagon-like Peptide (GLP) receptor agonist and is indicated as an adjunct to diet and exercise to improve glycemic control in adults with type 2. It is presently approved as solution for subcutaneous administration and as tablet for oral administration. Oral tablets are approved as 3 mg, 7 mg and 14 mg (Rybelsus) tablets which contains inactive ingredients magnesium stearate, microcrystalline cellulose, povidone and salcaprozate sodium (SNAC). The pharmaceutical composition as per present invention may contain therapeutically effective amount of semaglutide. The amount of semaglutide may range from 1 mg to 50 mg. More preferably, the amount of semaglutide may range from 1 mg to 20 mg. In one embodiment, the pharmaceutical composition of present invention contains 3 mg, 7 mg or 14 mg of semaglutide.

### Permeation Enhancers

Permeation enhancer are used to improve absorption of poorly permeable active pharmaceutical ingredients across the gastrointestinal tract. Permeation enhancer used in the composition as per present invention includes one or more medium chain fatty acid based permeation enhancers with aromatic functional group and one or more medium chain fatty acid based permeation enhancers with aromatic functional group.

The medium chain fatty acid based permeation enhancers with aromatic functional group may include but not limited to sodium 8-(2-hydroxybenzamido) caprylate, N-(10-[2-hydroxybenzoyl]amino) decanoic acid, N-(5-chlorosalicyloyl)-8-aminocaprylic acid. Sodium 8-(2-hydroxybenzamido) caprylate also known as Salcaprozate Sodium and is the sodium salt form of salcaprozate.

The amount of medium chain fatty acid based permeation enhancers with aromatic functional group may be in the range of 25 mg to 500 mg. In one embodiment the amount of medium chain fatty acid based permeation enhancers with aromatic functional group is 25 mg to 200 mg. In one embodiment, medium chain fatty acid based permeation enhancers with aromatic functional group is Sodium 8-(2-hydroxybenzamido) caprylate (Salcaprozate Sodium). The amount of Sodium 8-(2-hydroxybenzamido) caprylate (Salcaprozate Sodium) may be in the range of 25 mg to 500 mg. In an more specific embodiment the amount of Sodium 8-(2-hydroxybenzamido) caprylate (Salcaprozate Sodium) is 25 mg to 200 mg.

The medium chain fatty acid based permeation enhancers without aromatic functional group may include but not limited to sodium caprylate, sodium caprate, sodium hexanoate, sodium octanoate, sodium decanoate and sodium dodecanoate.

The amount of medium chain fatty acid based permeation enhancers without aromatic functional group may be in the range of 25 mg to 800 mg. In one embodiment, medium chain fatty acid based permeation enhancers without aromatic functional group is Sodium caprate or sodium caprylate. The amount of Sodium caprate or sodium caprylate may be in the range of 25 mg to 800 mg. In an more specific embodiment the amount of Sodium caprate or sodium caprylate is 200 mg to 500 mg.

In a one or more embodiments, the ratio of “medium chain fatty acid based permeation enhancers with aromatic functional group” to “medium chain fatty acid based permeation enhancers without aromatic functional group” in a pharmaceutical composition is 1:1 to 1:20. In a one or more preferred embodiments, the ratio of “medium chain fatty acid based permeation enhancers with aromatic functional group” to “medium chain fatty acid based permeation enhancers without aromatic functional group” in a pharmaceutical composition is 1:1 to 1:5.

### Basifiers

Basifier are compound that provide alkaline environment during and/or after dissolution of active ingredient. The basifiers may include but not limited to calcium carbonate, sodium carbonate, sodium bicarbonate, magnesium hydroxide, and aluminium hydroxide or combination thereof. The amount of basifier may be in the range of 25 mg to 600 mg. In one embodiment, basifier is Calcium carbonate.

The oral pharmaceutical composition as per present invention may further comprise one or more additional active ingredient. Non-limiting examples of additional active ingredients includes biguanides like metformin; glipizide or glyburide; DPP4 inhibitors like sitagliptin, alogliptin, linagliptin; SGLT2 inhibitors like dapagliflozin, canagliflozin, empagliflozin, ertugliflozin; rosiglitazone or pioglitazone; and repaglinide.

The oral pharmaceutical composition may be tablets, capsules, powders and granules, multi-particulate dosage forms and the likes. The multicompartment dosage form may be bilayer tablets, capsule-in-capsule, tablet-in capsule and any other dosage form. The pharmaceutical compositions can be formulated by any techniques known to or appreciated by a person skilled in the art. In a specific embodiment, pharmaceutical composition of present invention is prepared by direct compression method.

The oral pharmaceutical composition may further include optionally any one or a combination of one or more pharmaceutically acceptable excipients, such as but not limited to diluents, disintegrants, lubricating agents, binders, colorants, pigments, stabilizers, preservatives, antioxidants and solubility enhancers. Diluent may be selected from microcrystalline cellulose, lactose, mannitol, modified starches, dibasic calcium phosphate, any other diluent or combination thereof. Disintegrant may be selected from cross-linked polyvinylpyrrolidone, sodium starch glycolate, any other disintegrant or combination thereof. Binder may be selected from hydroxypropyl cellulose, hydroxypropyl methylcellulose, polyvinyl pyrrolidone, any other binder or combination thereof. Lubricants may be selected from calcium stearate, magnesium stearate, sodium stearyl fumarate, talc, any other lubricant or combination thereof.

Having described the invention with reference to the different embodiments of the invention, other embodiments will become apparent to one skilled in the art from consideration of the specifications.

The innovation is further defined by reference to the following examples. It will be apparent to those skilled in the art that many modifications to the composition may be practiced without departing from the scope of this invention.

### EXAMPLES

### Example—1

### Compositions:

| | | Quantity per |
|---|---|---|
| Sr No | Ingredient | tablet (mg) |
| 1 | Semaglutide | 7 |
| 2 | Sodium N-(8-(2- | 150 |
| | hydroxybenzoyl)amino)caprylate | |
| | (SNAC) | |
| 3 | Sodium caprate | 450 |
| 4 | Calcium carbonate | 100 |
| 5 | Sodium Starch Glycolate (SSG) | 100 |
| 6 | Magnesium stearate | 20 |
| | Total | 827 |

### Procedure:

1. All ingredients were weighed accurately.
2. SNAC, Sodium caprate, Calcium carbonate and SSG were mix in polybag for 3 min.
3. Semaglutide Powder added to above blend.
4. The above blend was passed through 40 mesh sieve.
5. The blend was lubricated using magnesium stearate for 3 min.
6. The Compression was performed using suitable punches.
7. The Tablets were packed in ALU-ALU blister.

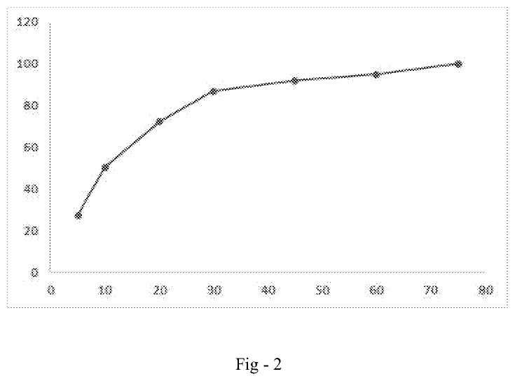

Dissolution profile of the composition as per example 1 was performed in Phosphate Buffer with pH 6.8 and is as shown in FIG. 2.

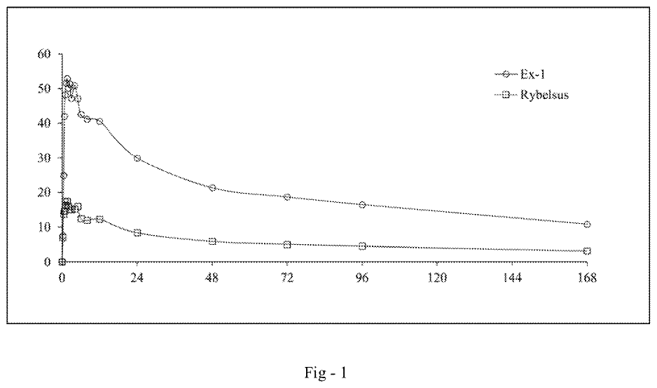

A Single dose fasted state 3 way cross over biostudies were performed for the composition of example 1 versus Rybelsus (reference) tablet in human volunteers. Result are shown in FIG. 1. Results shows surprisingly higher plasma concentration for the composition of example 1 compare to reference product Rybelsus.

| Formulation | Pilot study results (in healthy volunteers) |
|---|---|
| Rybelsus | AUC₀₋ₜ (ng × h/mL) values are 978.0 ± 1239.6 |
| | AUC_{0-∞} (ng × h/mL) values are 1470.9 ± 1822.5 |
| | Cₘₐₓ (ng/mL) values are 18.562 ± 25.549 |
| | Tₘₐₓ (h) values are 1.44 ± 0.72 |
| T1 -(Ex-1) | AUC₀₋ₜ (ng × h/mL) values are 3438.7 ± 4349.0 |
| | AUC_{0-∞} (ng × h/mL) values are 5007.6 ± 6375.7 |
| | Cₘₐₓ (ng/mL) values are 54.944 ± 67.907 |
| | Tₘₐₓ (h) values are 2.09 ± 1.54 |

### Example—2

| | | Quantity per |
|---|---|---|
| Sr No | Ingredient | tablet (mg) |
| 1 | Semaglutide | 7 |
| 2 | Sodium N-(8-(2- | 150 |
| | hydroxybenzoyl)amino)caprylate | |
| | SNAC) | |
| 3 | Sodium caprate | 300 |
| 4 | Calcium carbonate | 80 |
| 5 | Crospovidone | 80 |
| 6 | Magnesium stearate | 10 |
| | Total | 627 |

Procedure—Composition was prepared with analogous procedure similar to example 1.

### Example—3

| | | Quantity per |
|---|---|---|
| Sr No | Ingredient | tablet (mg) |
| 1 | Semaglutide | 7 |
| 2 | Sodium N-(8-(2- | 100 |
| | hydroxybenzoyl)amino)caprylate | |
| | (SNAC) | |
| 3 | Sodium caprate | 250 |
| 4 | Calcium carbonate | 60 |
| 5 | Sodium starch glycolate | 50 |
| 6 | Magnesium stearate | 10 |
| | Total | 477 |

Procedure—Composition was prepared with analogous procedure similar to example 1.

### Comparative Example A

### Composition

| | | |
|---|---|---|
| | | Quantity per |
| Sr No | Ingredient | tablet (mg) |

Intragranular

| | | |
|---|---|---|
| 1 | Sodium caprate | 450 |
| 2 | Sodium Lauryl Sulphate | 10 |
| | (SLS) | |
| 3 | Sodium starch glycolate | 150 |
| | (SSG) | |
| 4 | Povidone | 30 |
| 5 | Semaglutide | 7 |
| 6 | Water | QS |

Extragranular

| | | |
|---|---|---|
| 6 | Sodium starch glycolate | 100 |
| | (SSG) | |
| 7 | Magnesium stearate | 10 |
| | Total | 757 |

### Procedure

1. All ingredients were weighed accurately.
2. Ingredients 1 to 5 were sifted in 40 #sieve.
3. The powder mixture was mixed in polybag for 5 min.
4. Approximately 3 mL water added drop wise to achieve granulation endpoint.
5. The resulting wet granules were dried in hot air oven for 10 hours at 45 degrees Celsius.
6. The dry granules were passed through 40 #sieve.
7. Mg stearate and sodium starch glycolate were added as extra granular part and mixed for 5 minutes.
8. The resulting blend was compressed using suitable punches.

A Single dose fasted state 3 way cross over biostudies were performed for the composition of comparative example A versus Rybelsus (reference) tablet in human volunteers. Result are shown in FIG. 3. Results shows very low plasma concentration for the composition of comparative example A compare to reference product Rybelsus 7 mg.

I/We claim:

1. A pharmaceutical composition for oral administration comprising
a. a therapeutically effective amount of semaglutide or a pharmaceutically acceptable salt thereof,
b. 25 mg to 200 mg of salcaprozate sodium, and
c. 25 mg to 800 mg of sodium caprate or sodium caprylate or combinations thereof.

2. The pharmaceutical composition as claimed in claim 1, wherein said composition comprises 1 mg to 20 mg of semaglutide.

3. The pharmaceutical composition as claimed in claim 1, wherein said composition comprises 3 mg, 7 mg or 14 mg of semaglutide.

4. The pharmaceutical composition as claimed in claim 1, wherein the ratio of salcaprozate sodium to “sodium caprate or sodium caprylate or combinations thereof” in the pharmaceutical composition is 1:1 to 1:20.

5. The pharmaceutical composition as claimed in claim 1, wherein the ratio of salcaprozate sodium to “sodium caprate or sodium caprylate or combinations thereof” in the pharmaceutical composition is 1:1 to 1:5.

6. The pharmaceutical composition as claimed in claim 1, wherein said composition further comprises one or more basifier.

7. The pharmaceutical composition as claimed in claim 6, wherein the basifier is calcium carbonate.

8. The pharmaceutical composition as claimed in claim 1, wherein said composition further comprises crospovidone or sodium starch glycolate.

9. The pharmaceutical composition as claimed in claim 1, wherein said composition does not contains a binder.

10. The pharmaceutical composition as claimed in claim 1, wherein said pharmaceutical composition is a tablet.

11. The pharmaceutical composition as claimed in claim 10, wherein said tablet is prepared by direct compression method.

12. The pharmaceutical composition as claimed in claim 1, wherein said composition further comprises one or more additional active ingredient.

13. The pharmaceutical composition as claimed in claim 1, wherein AUC₀₋ₜ (ngxh/mL) for said pharmaceutical composition is 1.5 times or more compare to AUC₀₋ₜ (ngxh/mL) for reference product Rybelsus in single dose fasted study in human volunteers.

14. The pharmaceutical composition as claimed in claim 1, wherein Cmax (ng/mL) for said pharmaceutical composition is 1.5 times or more compare to Cmax (ng/ml) for reference product Rybelsus in single dose fasted study in human volunteers.
